(19) United States
(12) Patent Application Publication
(10) Pub. No.: US 2005/0080369 A1
(43) Pub. Date: Apr. 14, 2005

(54) Splint combined use cast absence for bone fracture fixing

(76) Inventor: Kyung-Jin Kim, Chungcheongnam-do (KR)

Correspondence Address: FAY, SHARPE, FAGAN, MINNICH & MCKEE, LLP, 1100 SUPERIOR AVENUE, SEVENTH FLOOR, CLEVELAND, OH, 44114, US

(21) Appl. No.: 10/500,430

(22) PCT Filed: Dec. 27, 2002
(86) PCT No.: PCT/KR02/02450

(30) Foreign Application Priority Data
Dec. 28, 2001 (KR) 10 2001 0087055

(51) Int. Cl. A61F 5/00

(52) U.S. Cl. 602/12

(57) ABSTRACT

Disclosed is a combined splint and cast for immobilizing the injured body part due to fracture, ligament rupture, dislocation or the like. In particular, the present invention relates to a combined splint and cast for immobilizing fractured bones, in which a splint and a cast used for a predetermined period of time until a swelling in the injured body part subsides are injection-molded to be combined into one. Therefore, the present invention can solve inconvenience of working with the conventional plaster caste member by using a hand fixing member, a connecting member an elbow fixing member of the combined splint and cast individually or cooperatively according to the use, for example, a short arm splint, a long arm splint or a hanging cast, minimize the sequelae, such as joint contracture by making possible early joint movement, and frequently correct and inaccurate reduction at its initial stage and malunion by taking intermediate inspections.

### TECHNICAL FIELD

The present invention relates to a combined splint and cast for immobilizing the injured body part due to fracture, ligament rupture, dislocation or the like. In particular, the present invention relates to a combined splint and cast for immobilizing fractured bones, in which a splint and a cast used for a predetermined period of time until a swelling in the injured body part subsides are injection-molded to be combined into one. Therefore, the present invention can solve inconvenience of working with the conventional plaster cast member by using a hand fixing member, a connecting member and an elbow fixing member of the combined splint and cast individually or cooperatively according to the use, for example, a short arm splint, a long arm splint or a hanging cast, minimize the sequelae, such as joint contracture by making possible early joint movement, and frequently correct an inaccurate reduction at its initial stage and abnormal fixation by taking intermediate inspections.

### BACKGROUND ART

In general, when bones are fractured, cracked, or ligaments are lengthened or ruptured, an orthopedic splint needs to be applied for the purpose of tightly supporting and immobilizing the injured joints and muscles partially or entirely.

Material used for the splint is flexible enough to be easily molded to conform to the curved shape of the injured body part, requires a proper amount of cure time, be easy to handle, and has mechanical strength high enough to maintain the structural strength after the cure. One of widely used methods using the qualified material is to wind a plaster bandage on the injured body part. Since such plaster bandage has high flexibility before solidification such that it can be easily molded to conform to the shape of the injured body part, requires a proper amount of cure time and work time, and has high mechanical strength, it has been widely used for a long time.

The plaster bandage, however, has a problem in that it has to be wound on the injured body part in sufficient thickness, thereby taking lots of time to be wound. To avoid the problem, presently, there has been developed a splint in which wet cure-type one component polyurethane resin is coated on a plaster bandage, glass fiber or synthetic fabric, and the resultant coating is repeatedly wound into multi layers, thereby reducing the time taken to be used. When the splint constructed as above is applied to the curved body part, such as the elbow, the knee, the heel or the like, it has to be bent to conform to the angle of the injured body part. Inevitably, it gets crumpled on the curved part. Further, since it is made in a long rectangular form, it fails to closely wrap the injured body part in accordance with the thickness of the curved shape, e.g., the wrist, arm, ankle and calf. In this case, the splint gets loose from the injured body part and fails to tightly support the injured body part, thereby badly affecting the fractured part.

Furthermore, the method using the plaster bandage has problems in that since the work process is complicated and the plaster bandage is wound over the injured body part, mold or infectious bacteria may grow. In addition, the process of cutting the cast by using a saw to remove the cast raises the dust.

In recent years, to obviate those disadvantages, Utility Model Publication No. 1995-13343 entitled a cast member for fracture patients and Utility Model Publication No. 2001-0016563 entitled a splint structure disclose a method of molding thermoplastic resin into a thin sheet and adhering the inner surface of a sponge to the resin. In those disclosures, a splint is put into an oven and gets softened at the temperature of 90° C. or so. The splint is put on the injured body part and closely attached to the injured body part along the curved surface by being pressed with hands. Since the soften splint made of thermoplastic resin gets cool during this forming process, it cannot be closely attached to the injured body part at once completely. For the reason, the splint has to be put into the oven more than one time to be softened and cooling time of about 20 minutes is required after the forming process is finished so as for the splint to become cold completely. Thus, the splint causes inconvenience in application and takes lots of time, resulting in poor practicality.

A short arm splint is used to immobilize the hand. A long arm splint is used to immobilize the region from the wrist to a portion below the elbow (the middle portion of the humerus). A hanging cast is used to immobilize the region from the wrist to the middle portion of the humerus. Before a cast is applied, the splints are used for a predetermined period of time to lessen the swelling in the injured body part. In the conventional art, since the splint and the cast are separately applied, inconveniently materials for the splint and the cast are separately prepared and applied.

Moreover, when the cast is applied on the injured body part, early joint movement is difficult to be done during the cast period, and inaccurate reduction and abnormal fixation cannot be checked through intermediate inspections.

### DISCLOSURE OF INVENTION

Accordingly, the present invention is directed to a combined splint and cast for fixing fractured bones that substantially obviates one or more problems due to limitations and disadvantages of the related art.

An object of the present invention is to provide a combined splint and cast for fixing fractured bones which can fast and easily fix the fractured bones by injection-molding a hand fixing member, a connecting member, and an elbow fixing member of various sizes according to body measurements and using the respective members individually or cooperatively according to the use, for example, a short arm splint, a long arm splint or a hanging cast, and reduce the time consumed to apply the cast and decrease the cost for the cast application by combining a splint and a cast into one and omitting the intermediate process of applying the plaster cast.

Another object of the present invention is to provide a combined splint and cast for fixing fractured bones which can obtain a precise immobilization effect by eliminating difference in cast application skill between surgical operators and assistants, and prevent side effects, such as contamination caused by mold or infectious bacteria on the cast portion by providing a plurality of ventilation holes on the respective members so as for air to be smoothly circulated in the injured body part, differently from the conventional method in which the cast entirely encloses the injured body part.

A further object of the present invention is to provide a combined splint and cast for fixing fractured bones which can minimize joint contracture and reduce duration of treatment by making early rehabilitation and early joint movement possible during the cast period, achieve a precise union by performing intermediate checks on initial inaccurate reduction or abnormal fixation, and make a patient feel comfortable during movement by using the respective members made of light synthetic resin.

A still further object of the present invention is to provide a combined splint and cast for fixing fractured bones which can give aesthetic effects by injection molding the respective members to be applied to the right and left hands and at the same time imparting various colors to the respective members.

To achieve these objects and other advantages, there is provided a combined a combined splint and cast for immobilizing the injured body part due to fracture, ligament rupture, dislocation or the like, the combined splint and cast comprising: a hand fixing member including a dorsum manus member for wrapping the back of the hand and the wrist and a palm member for supporting a palm of the hand, the dorsum manus and palm members forming a pair; a connecting member including right and left connecting members which form a pair for wrapping the region from the wrist to below the elbow; and an elbow fixing member for immobilizing the region from the elbow to the middle portion of the humerus, wherein the hand fixing member, the connecting member and the elbow fixing members are used separately or cooperatively by being fastened with bolts.

It is to be understood that both the foregoing general description and the following detailed description of the present invention are exemplary and explanatory and are intended to provide further explanation of the invention as claimed.

### BRIEF DESCRIPTION OF THE DRAWINGS

Further objects and advantages of the invention can be more fully understood from the following detailed description taken in conjunction with the accompanying drawings in which:

FIG. 1 is a front elevation view illustrating the human skeletal system;

FIG. 2 is an exploded perspective view illustrating a combined splint and cast according to the present invention;

FIG. 3 is a perspective view illustrating a dorsum manus member in use according to the present invention;

FIG. 4 is a perspective view illustrating a hand fixing member in use according to the present invention;

FIG. 5 is a perspective view illustrating a left connecting member in use according to the present invention in use;

FIG. 6 is a perspective view illustrating a connecting member in use according to the present invention;

FIG. 7 is a perspective view illustrating an elbow fixing member in use according to the present invention; and

FIG. 8 is a perspective view illustrating an embodiment according to the present invention.

### BEST MODE FOR CARRYING OUT THE INVENTION

The present invention will now be described in detail in connection with preferred embodiments with reference to the accompanying drawings. For reference, like reference characters designate corresponding parts throughout several views.

The following detailed description will present a preferred embodiment of the invention in reference to the accompanying drawings.

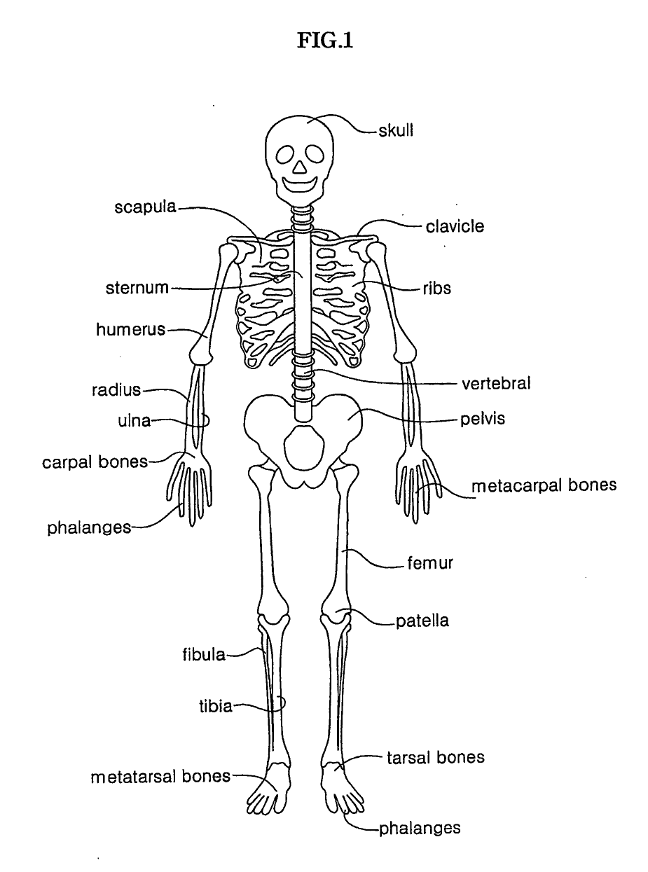

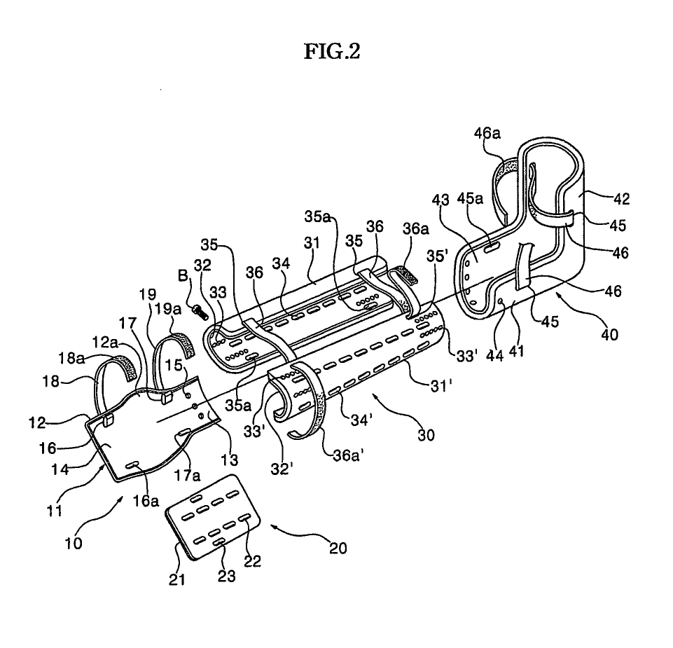

FIG. 1 is a front elevation view illustrating the human skeletal system, and FIG. 2 is an exploded perspective view illustrating a combined splint and cast according to the present invention. The combined splint and cast shown in FIG. 2 is used in fracture of the upper limb, which occurs owing to impact against the upper limb bones, indirect stress, or wound owing to a fall from a vaulting horse, horse or bicycle.

Fracture causes severe pain, and if a damaged part is pressed or contacted, more severe pain is felt. Fractured part swells owing to bleeding since soft tissues are damaged, and would be transformed in appearance if it is severely fractured. In order to perform a surgical operation via fixation and reduction of fractured bones, the combined splint and cast has several members which are used according to body parts. The members include a hand fixing member 10 having a dorsum manus member 11 and a palm member 20 for immobilizing the region from metalcarpal bones (regions including back side and palm of the hand) to carpal bones (a portion above the wrist), a connecting member 30 connected to the hand fixing member 10 at one end thereof for immobilizing the region from above the wrist below the elbow (the radius and ulna) and an elbow fixing member 40 connected to the other end of the connecting member 30 for immobilizing the region from the elbow to the middle portion of the humerus. These members can be used separately or cooperatively.

The hand fixing member 10 is used as a short arm splint in case of fracture in metacarpal bones and/or carpal bones.

Metacarpal bones and carpal bones are most frequently fractured when a user falls during exercise on a movable equipment or horse. As shown in FIG. 1, the metacarpal bones mean five bones in a palm connected to phalanges (fourteen bones of fingers), and the carpal bones mean eight bones in the wrist, i.e., scaphoid bone, lunate bone, triquetrum bone, pisiform bone, trapezium bone, trapezoid bone, capitate bone and hamate bone. When the metacarpal bones or the carpal bones are damaged, a splint is primarily used, in which the splint is fixed after selecting a hand fixing member 10 of a size corresponding to a specific body part (hand) of a patient.

In the hand fixing member 10, the dorsum manus member 11 and the palm member 20 make a pair to wrap the region from the back side of the hand (metacarpal bones) to above the wrist (carpal bones). Various sizes of the hand fixing members are injection molded according to hand sizes based upon the standardized research result on human body dimensions. Also, right and left hand fixing members 10 are molded to meet respective conditions depending on the right and left hands.

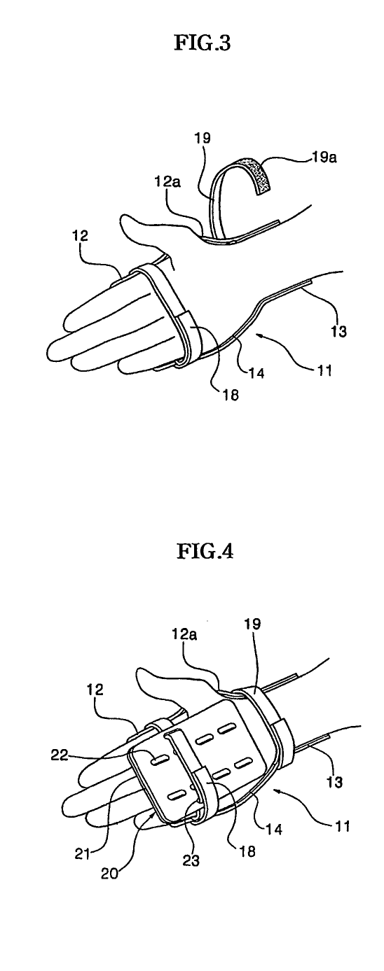

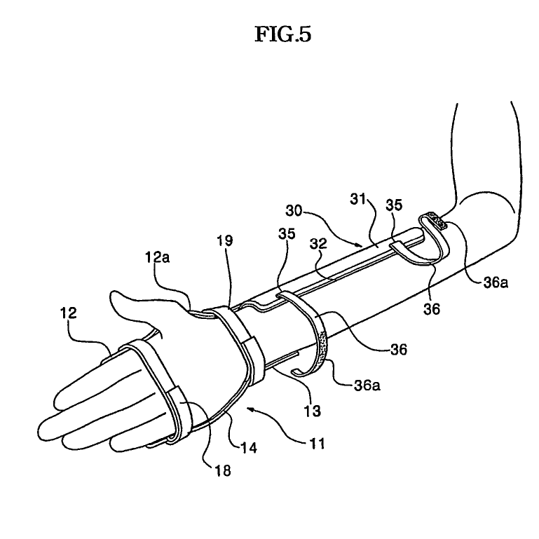

The dorsum manus member 11 forms a dorsum manus plate 12 in a substantially identical shape with the back side of the hand to wrap the back side of the hand as shown in FIGS. 3 and 4. The dorsum manus plate 12 forms an extension 13 projected to the wrist so that the inner face of the plate 12 closely contacts the wrist.

A buffering material 14 made of sponge is adhered to the inner face of the dorsum manus member 11 to wrap the back side of the hand and the wrist.

The inner face of the extension 13 of the dorsum manus member 11 has a plurality of bolt holes 15, and one end of the connecting member 30 (which will be described later) is closely contacted by the extension 13 such that the dorsum manus member 11 is coupled to the connecting member 30 with bolts B inserted through the bolts holes 15. The dorsum manus plate 12 of the hand fixing member 10 is integrally formed with a support 12a upwardly projected from a portion in which the thumb is placed. The support 12a functions to securely fix a metacarpophalangeal joint of the thumb under navicular bone fracture which frequently occurs in ski or racket games.

The connecting member 30 coupled with the extension 13 of the dorsum manus member 11 can be fixed in various manners, for example, via an instantaneous adhesive.

In order to securely wrap the back side of the hand as in FIG. 3, the dorsum manus member 11 has four holes 16, 16a; 17, 17a in four corners of the dorsum manus plate 12. One end of a fastener tape 18 is fixed to the hole 16, and the other end of the fastener tape 18 is passed through the opposite hole 16a such that the dorsum manus member 11 is fixed to the back side of the hand. Then, a fastener tape 19 is also fixed to the holes 17 and 17a in the same manner to complete the splint application.

The dorsum manus 11 shown in FIG. 3 is used until a swelling in an affected part subsides. FIG. 4 shows use of the palm member 20 in which a cast is worn on the entire parts of the hand.

The palm member 20 is used in cooperation with the dorsum manus member 11 when the palm wears a cast. The palm member 20 is planar shaped to be closely contacted by the palm face, and has a buffering material 21 for being smoothly contacted by the palm. A plurality of ventilation holes 22 are formed in the palm member 20 so that external air can contact the palm via the ventilation holes 22, thereby preventing contamination of mold or infectious bacteria.

The palm member 20 has holes 23 in the upper and lower portions so that the fastener tape 18 is inserted into the same to couple the palm member 20 with the dorsum manus member 11. In order to closely contact the palm member 20 to the dorsum manus member 11, the other end of the fastener tape 18 passes through the holes 23 of the palm member 20 to enter the hole 16a of the dorsum manus member 11. Alternatively, two fastener tapes 18 can be provided in both of the upper and lower portions of the dorsum manus member 11 to fix the palm member 20. The fastener tape 18 has an adhesive part 18a which is formed from the middle portion of the fastener tape 18 to the distal end or the other end so that the distal end of the fastener tape 18 is fastened to the middle portion thereof to couple the dorsum manus member 11 with the palm member 20 as shown in FIGS. 3 and 4.

Also, the fastener tape 18 for fastening the dorsum manus member 11 can be substituted by other fastening materials, such as a belt or a string in order.

The connecting member 30 is a long arm cast for generally immobilizing the arm from proximal joints of fingers to one third of a distal part of the arm. The connecting member 30, the hand fixing member 10, and the elbow fixing member 40 are used for immobilizing the arm in case of radius and ulna fracture.

The arm has two bones from the elbow to the wrist, i.e., radius and ulna. The radius is a bone having a length of about 21 cm adjacent to the thumb, in which the lower portion of the radius is thicker. The ulna is a bone having a length of about 22 cm adjacent to the minimus.

Radius and/or ulna fracture takes place when the patient touches the ground and the like with the wrist pulled back or falls and touches the ground with the wrist pulled forward. The radius and ulna are connected to each other in the wrist and the elbow respectively via radioulnar joints, and rotate about the radial head. The ulna is substantially linear, whereas the radius is curved defining an angle of about 6 to 9 degrees. In forearm fracture, it is difficult to reduce and maintain the fractured parts since muscles react differently according to the position of the fractured part, or to perform rotational motion in the case of malunion or excessive callus (where a large quantity of ooze leaks from the bones).

In the forearm bones, the ulna and the radius may be individually or simultaneously broken, and fracture or dislocation may take place. In particular, in Monteggia Fracture where an ulna proximal part is fractured and the radius head is disarticulated or Galeazzi Fracture where an ulna distal part is fractured and the lower radioulnar joint is disarticulated, a child can have a relatively satisfactory result via reposition and plaster fixation by hands. However, an adult cannot expect a satisfactory result without an operation since rotation trouble may take place or premature arthritis occurs due to malunion. Fracture of a forearm is more frequent in children than in adults, and can be diagnosed via front, rear and side pictures. If the patient feels a severe pain or uncomfortable in the joint, four-side photographing to the wrist and elbow joints is necessary to inspect whether the patient has linear fracture or dislocation. Further, fracture of an arm essentially needs periodic radionuclei study after fixed with a cast since fractured bones may easily be displaced or the joint may readily disarticulate since bones move even in movement of fingers.

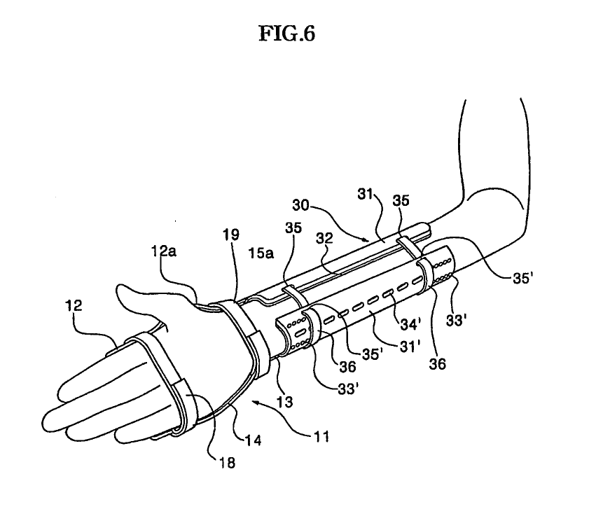

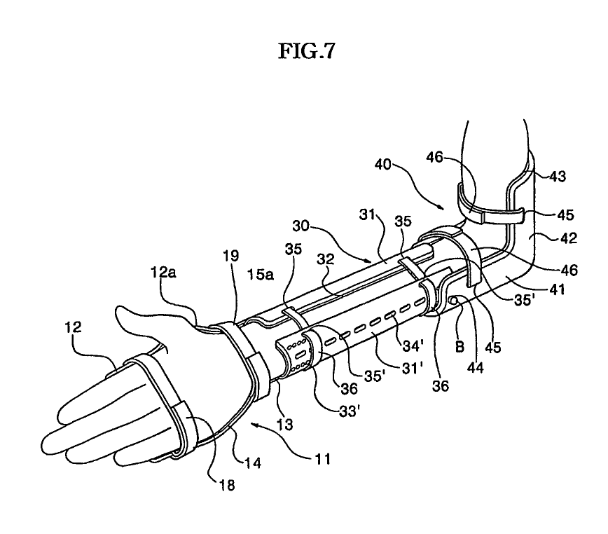

As shown in FIGS. 5 to 8, the connecting member 30 has the left and right connecting members 31 and 31′ which form a pair having a substantially identical structure with each other for wrapping the region from proximal joints of fingers to one third of a distal part of the arm. The connecting member 30 is injection molded based upon the research result of standardized human body dimensions to have a substantially identical shape with a body part.

The connecting member 30 is formed in such a manner as to wrap the entire portions of the radius and the ulna from the wrist (carpal bones) via the radius and the ulna to a portion below the elbow. The left connecting member 31 is shaped to wrap the arm from the wrist to a portion below the elbow and has an inside buffering material 32 as shown in FIG. 5.

The left connecting member 31 has a plurality of bolt holes 33 in inner peripheries of the front and rear portions at equal intervals so that the combined splint and cast can be adjusted in length. The leading portion of the left connecting member 31 is closely contacted by the extension 13 of the dorsum manus member 11 such that the left connecting member 31 is coupled to the dorsum manus member 11 with the bolts B inserted through the bolt holes 33. A plurality of ventilation holes 34 are formed at opposite lateral sides of the left connecting member 31 so that external air can readily circulate via the ventilation holes 34 to prevent contamination caused by mold or infectious bacteria. The upper and lower holes 35 and 35a are formed in the front and rear ends of the left connecting member 31, and two fastener tapes 36 have one ends fixed to the upper holes 35. The other end of each of the fastener tapes 36 is passed through the upper and lower holes 35′ and 35a′ in the right connecting member 31′ connected to the left connecting member so as to securely wrap radius and ulna parts.

The right connecting member 31′ has a substantially identical structure to the left connecting member 31, and the left and right connecting members 31 and 31′ are symmetric to each other.

The other end of the each fastener tape 36 is inserted into the lower hole 35a of the left connecting member 31 while wrapping an outer periphery of the right connecting member 31′ past through the upper and lower holes 35′ and 35a′ of the right connecting member 31′. Then, the other end of the each fastener tape 36 is attached to an adhesive part 36a in an outer portion of the fastener tape 36 to securely contact the left and right connecting members 31 and 31′

Alternatively, four fastener tapes 36 can be provided in the upper and lower portions in the front and rear ends of the left connecting member 31 to separately fix the left and right connecting members.

The elbow fixing member 40 immobilizes the region below the elbow to the middle portion of the humerus. The elbow fixing member 40 is used in the long arm splint to couple with one end of the connecting member 30 to securely fix the elbow part.

The arm generally indicates forearm, but can be divided into a forearm from the elbow joint to the hand and the upper arm from the elbow joint to the shoulder. The upper arm has one generally cylindrical bone, i.e., a humerus which has a planar portion adjacent to the elbow. The humerus is swollen at the shoulder to form a round bone head and meets the concave glenoid cavity of a scapula so that two bones are connected to define a shoulder joint while maintaining stability between the projected surface and the concave surface. The humerus is attached with muscles for moving the arm or shoulder, and also serves as a passage of the upper arm artery and vein, an ulna nerve, a radius nerve, a median nerve and a musculocutaneous nerve leading to the hand or forearm. Therefore, when blood vessels or nerves are damaged owing to humerus fracture, an emergency operation is necessary.

When displacement occurs after fracture, it is difficult to assemble spicules in position since the spicules move to a proximal or distal position or an inside or outside owing to a fractured part, damaged direction, the intensity of force, the upper arm muscles or the weight of a broken arm. The radius nerve for extending a wrist or fingers (i.e., folding the wrist or fingers toward the dorsum manus) passes near the humerus at about one third of a distal point of the humerus. Since the wrist or fingers are not spread if the nerve is caught between the spicules owing to fracture at this point, it is necessary to pay a specific attention during emergency measure or operation after being damaged.

Diagnosis can be readily performed via simple radionuclei study on front, rear or lateral photographing. However, in case of shattered, inclined or spiral fracture, it is necessary to have a four-side photographing on the wound. When the vessels or nerves are injured together with bones, it is necessary to frequently examine the injured body part within early three days after fracture. Emergency operation would be performed if necessary.

The elbow fixing member 40 has a horizontal wall 41 in which the elbow is seated and a vertical wall 42 folded perpendicular from the horizontal wall 41 for wrapping the region from the elbow to the middle portion of the humerus so as to wrap the region from the arm to the middle portion of the elbow humerus. The elbow fixing member 40 also has a buffering material 43 formed in the inner face thereof and a plurality of bolt holes 44 formed in the inner peripheral surface of a leading end thereof, which are fastened into the bolt holes 33 in the rear end of the extension 30 via the bolt B.

A plurality of holes 45 and 45a are formed in opposed the upper portions of the horizontal and vertical walls 41 and 42, and two fastener tapes 46 are provided. One end of each of the fastener tapes 46 is fixed to each of the holes 45, and the other end of the each fastener tape 46 is passed through each of the holes 45′. After adjusting the length of the each fastener tape 46, the other end thereof is attached to a fixed fastening face 46a of the each fastener tape 46.

The hand fixing member 10, the connecting member 30 and the elbow fixing member 40 can be used cooperatively by being fastened via the bolts B or separately used.

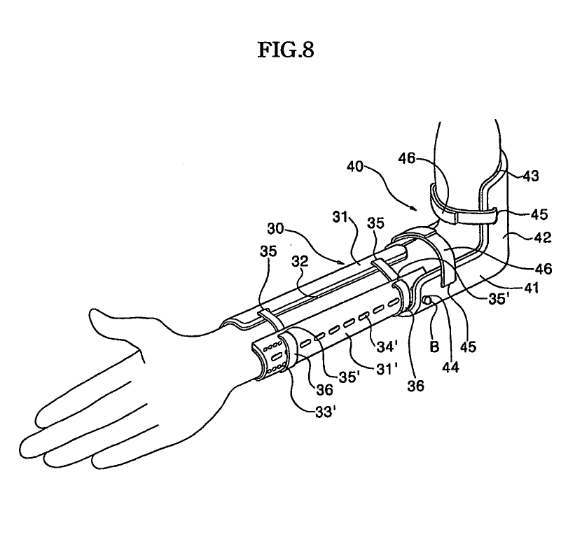

In a hanging cast for immobilizing the arm from above the wrist (carpal bones) to one third of a distal part in simple fracture of humerus cadre, the fractured part is fixed by using the connecting member 30 and the elbow fixing member 40 as shown in FIG. 8.

In operation of the invention having the above construction in reference to FIGS. 2 to 8, the hand fixing member 10, the connecting member 30 and the elbow fixing member 40 are injection molded from synthetic resin based upon the research result of standardized body dimensions and divided into left and right pieces so that they can be used corresponding to body sizes of the patient. Since the invention provides the combined splint and cast, it is unnecessary to prepare an additional splint material for fixing the arm.

In the case of the short arm splint, the hand is passed through the extension 13 of the hand fixing member 10, and the back side of the hand is closely pressed against the buffering material 14 in the inner face of the hand fixing member 10. As a cotton bandage is inserted between the buffering material 14 and the hand to prevent any movement between them, the hand fixing member 10 is fixed to the metacarpal bones (dorsum manus) and the carpal bones (wrist) via the fastener tapes 18 and 19. If the swelling in the hand subsides more or less after a certain time period, the palm member 20 is placed on the palm regarding the condition of the metacarpal bones. Then, the palm member 20 is fixed via the fastener tape 18 of the dorsum manus member 11.

In the case of the long arm splint, the hand fixing member 10, the connecting member 30 and the elbow fixing member 40 are used together. The hand is primarily placed on the dorsum manus member 11 as set forth above, the left connecting member 31 is fastened into the bolt holes 15 of the dorsum manus member 11 via the bolt B at a length corresponding to the body size of the patient. As described above, the cotton bandage is used to prevent movement of the left connecting member 31, and then the fastener tapes 36 are wound to fix the left connecting member 31.

The bolt holes 44 in the horizontal wall 41 of the elbow fixing member 40 are matched to the rear bolt holes 33 in the left connecting member 31, and then the holes 44 and 33 are fastened via the bolt B and fixed by means of the fastener tapes 46. When the swelling subsides more or less after a certain time period, the palm member 20 and the right connecting member 31′ are separately coupled according to the condition of the patient to place a cast on the patient.

In the case of the hanging cast, the connecting member 30 is used in cooperation with the elbow fixing member 40. The connecting member 30 is primarily fixed from the portion above the wrist (carpal bones) to the portion below the elbow, and the elbow fixing member 40 is fastened to the rear end of the connecting member 30 with the bolt B after adjustment of the length. The fastener tapes 46 are wound and fixed around the arm and the humerus to completely place the cast on the patient.

According to the present invention as set forth above, the cast members are injection molded by selecting certain strength of materials. Since the cast members are provided according to body sizes, they can be systematically worn by the patient. Furthermore, the cast members are lighter to reduce inconvenience when the patient moves.

Industrial Applicability

As set forth above, the present invention relates to the combined splint and cast for immobilizing the injured body part due to fracture, ligament rupture, dislocation or the like. The hand fixing member, the connecting members and the elbow fixing member can be injection molded based upon the research result on standardized body dimensions so that the members can be used cooperatively or separately according to the damaged condition of the patient, thereby saving cast operation time and related cost.

The present invention eliminates the variation in cast application skill between surgical operators and assistants. Since the respective members can be separated and have the plurality of ventilation holes, the invention can prevent side effects, such as mold or bacteria contamination observed in a conventional cast.

The present invention provides the cast members made of light synthetic resin to reduce inconvenience that the patient feels during movement.

The present invention combines the splint and cast members as well as systematically applies the cast operation so that the wound can be intermediately inspected while the patient wears the cast, thereby improving the condition of bonded bones.

In the present invention, the patient can wear a half-splint after an operation and the wound can be intermediately inspected, resultantly enabling early recovery via early rehabilitation.

Moreover, the present invention imparts various colors to the respective members thereby giving aesthetic effects.

1. A combined splint and cast for immobilizing the injured body part due to fracture, ligament rupture, dislocation or the like, the combined splint and cast comprising:
a hand fixing member including a dorsum manus member for wrapping the back of the hand and the wrist and a palm member for supporting a palm of the hand, the dorsum manus and palm members forming a pair;
a connecting member including right and left connecting members which form a pair for wrapping the region from the wrist to below the elbow; and
an elbow fixing member for immobilizing the region from the elbow to the middle portion of the humerus,
wherein the hand fixing member, the connecting member and the elbow fixing members are used separately or cooperatively by being fastened with bolts.

2. The combined splint and cast as defined in claim 1, wherein the hand fixing member, the connecting member and the elbow fixing member are injection molded.

3. The combined splint and cast as defined in claim 1, wherein the hand fixing member includes:
a dorsum manus member having a dorsum manus plate of a shape substantially identical to the back side of the hand for wrapping the back side of the hand and an extension extended from one end of the dorsum manus to a wrist part for being closely contacted by the wrist;
a buffering material arranged in the inner face of the dorsum manus member for wrapping the back side of the hand;
a plurality of bolt holes formed in the inner face of the extension, wherein one end of the connecting member is in close contact with the inner face of the extension and the hand fixing member is coupled to the connecting member with bolts inserted through the bolt holes;
a plurality of holes formed in the upper and lower portions of the front and rear ends of the dorsum manus plate; and
a fastener tape with one end fixed to any one of the holes and the other end fixed to the palm member for adjusting the contact condition between the dorsum manus member and the palm member.

4. The combined splint and cast as defined in claim 3, wherein the dorsum manus member has a support projected from the upper portion of the dorsum manus plate in which the thumb is seated for immobilizing the thumb in case of fracture in scaphoid bone.

5. The combined splint and cast as defined in claim 1 or 3, wherein the palm member is configured in a planar form for immobilizing the palm, and includes:
a buffering material arranged in the inner face of the palm member for smoothly wrapping the palm;
a plurality of ventilation holes formed at opposite lateral sides of the palm member for allowing air circulation; and
holes formed in the upper and lower portions of the palm member for allowing the other end of the fastener tape which is fixed to the dorsum manus member to be inserted therethrough.

6. The combined splint and cast as defined in claim 1, wherein the connecting member is formed of substantially identical left and right connecting members for wrapping both lateral sides of the arm from the wrist to below the elbow, and includes:
a buffering material arranged in the inner face of the connecting member;
a plurality of bolt holes formed in the inner peripheries of the front and rear portions at equal intervals for allowing length adjustment such that the connecting member is coupled to the dorsum manus member with bolts inserted through the bolt holes;
a plurality of ventilation holes formed at opposite lateral sides thereof in a longitudinal direction in the inner face thereof;
a plurality of holes formed in the upper portions of the front and rear ends of the left and right connecting members; and
a fastener tape with one end fixed to any one of the holes and the other end inserted into a remaining one of the holes for securely wrapping radius and ulna parts.

7. The combined splint and cast as defined in claim 1, wherein the elbow fixing member includes:
a horizontal wall in which the elbow is seated;
a vertical wall bent from the horizontal wall for wrapping the region from the elbow to the middle portion of the humerus;
a buffering material arranged in the inner face thereof;
a plurality of volt holes formed in the inner peripheral surface of a leading end of the horizontal wall such that the ankle fixing member is coupled to the rear end of the connecting member with bolts inserted through the bolts holes;
a plurality of holes formed in the upper portions of the horizontal and vertical walls; and
a fastener tape with one end fixed to any one of the holes and the other end coupled with an opposed one of the holes for securely immobilizing the elbow and the humerus.
